(19) United States
(12) Patent Application Publication
(10) Pub. No.: US 2025/0134554 A1
(43) Pub. Date: May 1, 2025

(54) SURGICAL INSERT INSTRUMENT

(71) Applicant: Russell Todd Nevins, Las Vegas, NV (US)

(72) Inventor: Russell Todd Nevins, Las Vegas, NV (US)

(21) Appl. No.: 18/935,388

(22) Filed: Nov. 1, 2024

Related U.S. Application Data
(60) Provisional application No. 63/595,299, filed on Nov. 1, 2023.

(51) Int. Cl. A61B 17/56; A61B 90/00

(52) U.S. Cl. CPC A61B 17/56; A61B 90/03

(57) ABSTRACT

Disclosed is a surgical instrument having a handle with an elongate member extending from the handle. The handle may be coupled to the elongate member at a first portion of the elongate member. The surgical instrument may also include a tip extending from the elongate member such that the tip may be coupled to a second portion of the elongate member. The tip may include a top surface and a bottom surface extending from a terminating end of the tip, and the top surface and the bottom surface may form a converging angle. The tip may also include an abutment that may be substantially perpendicular to the bottom surface.

### CROSS-REFERENCE TO RELATED APPLICATIONS

This application claims the benefit of U.S. Provisional Application No. 63/595,299, filed Nov. 1, 2023, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not applicable.

### BACKGROUND

### 1. The Field of the Present Disclosure

The present disclosure relates generally to surgical systems and methods of facilitating the efficiency and accuracy of inserting a surgical prostheses into a prostheses holder, cup or receiver.

### 2. Description of Related Art

The present disclosure relates generally to surgical systems and methods of facilitating the efficiency and accuracy of implanting surgical prostheses using surgical tool to facilitate proper placement of the prostheses.

In traditional implant surgeries, e.g., knee replacements, a surgeon will utilize a implant holder to receive a tibial implant, such that the tibial implant may be removably engaged with the implant holder.

The tibial implant and the implant holder may typically be engaged via a tongue-and-groove type of engagement, securing the implant to the implant holder when properly seated, but allowing the implant to be disengaged from the implant holder when desired.

However, in practice, the implant may not be fully engaged with and secured to the implant holder if the corresponding tongue-and-groove connection is not fully and properly engaged. If the implant is not inserted into the implant holder at the proper, predetermined angle, then the tongue and groove may only be partially engaged, leaving the implant improperly seated to the implant holder, which may create instability and/or breakage of the implant and/or implant holder.

The features and advantages of the present disclosure will be set forth in the description which follows, and in part will be apparent from the description, or may be learned by the practice of the present disclosure without undue experimentation. The features and advantages of the present disclosure may be realized and obtained by means of the instruments and combinations particularly pointed out in the appended claims. Any discussion of documents, acts, materials, devices, articles or the like which has been included in the present specification is not to be taken as an admission that any or all of these matters form part of the prior art base, or were common general knowledge in the field relevant to the present disclosure as it existed before the priority date of each claim of this application.

### BRIEF DESCRIPTION OF THE DRAWINGS

The features and advantages of the disclosure will become apparent from a consideration of the subsequent detailed description presented in connection with the accompanying drawings in which:

FIG. 1 is a perspective view of a surgical instrument of the present disclosure;

FIG. 2 is an exploded perspective view of an arm of the surgical instrument of FIG. 1;

FIG. 3 is side view of the surgical instrument of FIG. 1;

FIG. 4 is perspective view of an arm of the surgical instrument of FIG. 1;

FIG. 5 is perspective view of a handle of the surgical instrument of FIG. 1;

FIG. 6 is side view of a tip of the surgical instrument of FIG. 1;

FIG. 7 is perspective view of the tip of the surgical instrument of FIG. 1;

FIG. 8 is a top view of the tip of the surgical instrument of FIG. 1;

FIG. 9 is perspective view of an implant and implant holder of the present disclosure;

FIG. 10 is perspective view of the implant holder of FIG. 7;

FIG. 11 is perspective view of the surgical instrument of FIG. 1 engaged with the implant and implant holder of FIG. 7;

FIG. 12 is perspective view of another embodiment of an arm of the surgical instrument of FIG. 1;

FIG. 13 is top view of the arm of FIG. 12;

FIG. 14 is a side view of the arm of FIG. 12;

FIG. 15 is perspective view of another embodiment of an arm of the surgical instrument of FIG. 1; and

FIG. 16 is top view of the arm of FIG. 15.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

It must be noted that, as used in this specification and the appended claims, the singular forms “a,” “an,” and “the” include plural referents unless the context clearly dictates otherwise.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

The phrase “surgical instrument” as used herein, shall refer broadly to an surgical tool, a surgical implant, an operating or medical device, or any other object that may be used during the course of a medical procedure. The phrase “surgical instrument” as used herein, shall also refer to any known definition of that phrase that is either consistent with or not inconsistent with the definition in the forgoing sentence.

The term “tip” as used herein, shall refer broadly to an any portion of an object that includes a terminating end of said object. The term “tip” as used herein, shall also refer to any known definition of that term that is either consistent with or not inconsistent with the definition in the forgoing sentence.

The term “handle” as used herein, shall refer broadly to any portion of an object that may be grasped, held, and/or carried by a user of said object. The term “handle” as used herein, shall also refer to any known definition of that term that is either consistent with or not inconsistent with the definition in the forgoing sentence.

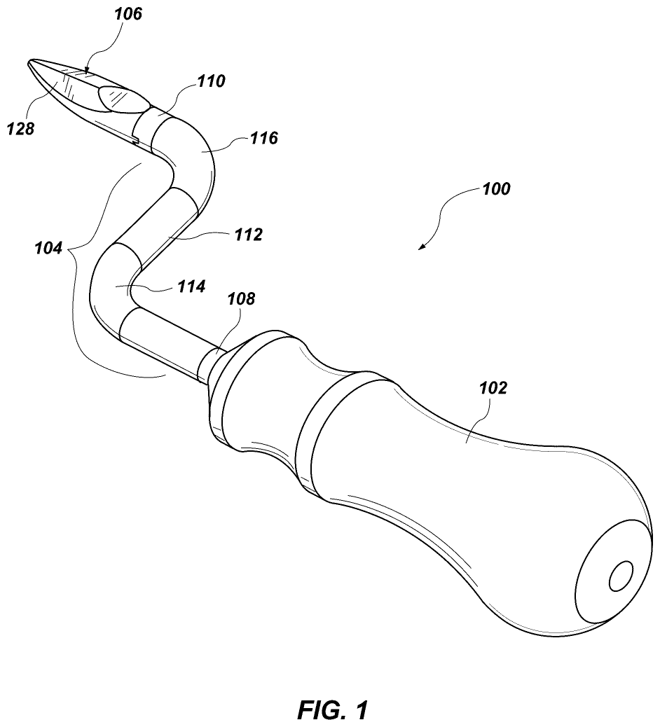

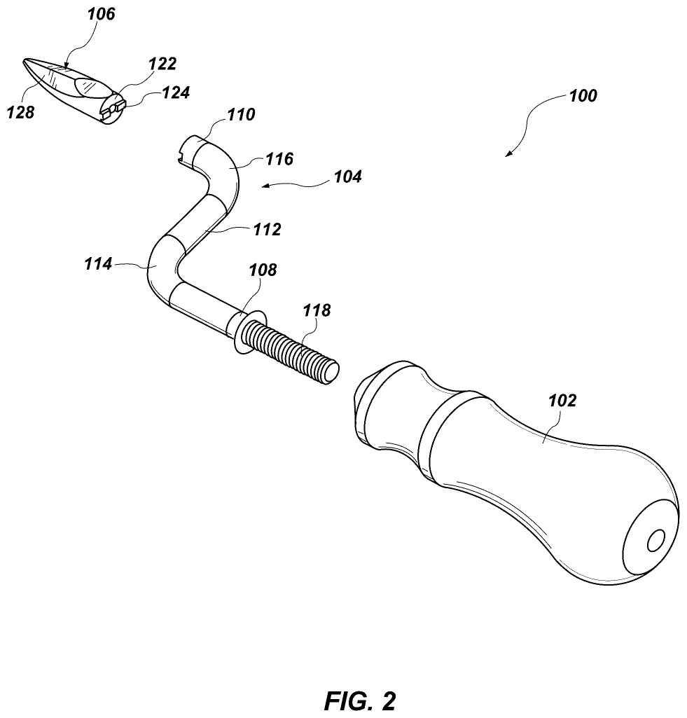

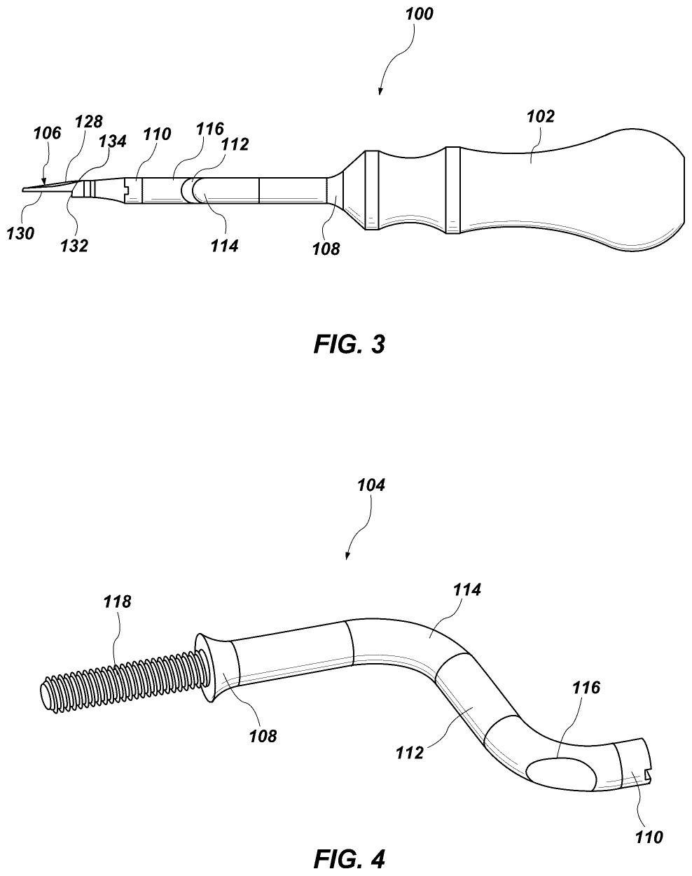

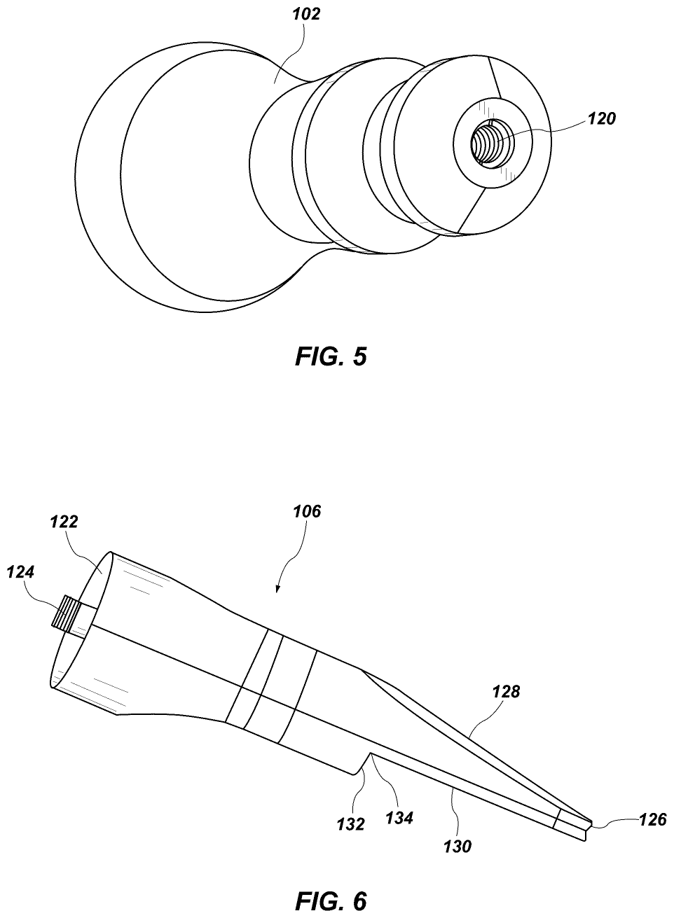

Referring now to FIGS. 1-6, a disclosed embodiment illustrates a surgical instrument 100, which can be used to facilitate or aid in the mounting or insertion of a surgical implant into an implant holder or cup.

The surgical instrument 100 may include a handle 102, an elongate member, or arm, 104 and a tip 106. The arm 104 may be fixed or removably coupled to the handle 102. The tip 106 may be fixed or removably coupled to the arm 104.

The arm 104 may have a first portion 108 that may removably coupled or fixed to the handle 102. The arm 104 may also have a second portion 110 that may be removably coupled or fixed to the tip 106. The arm 104 may also include an intermediate portion 112 that is positioned or located between the first portion 108 and the second portion 110.

The arm 104 may include a first bend 114 located or positioned between the first portion 108 and the intermediate portion 114. The arm 104 may also include a second bend 116 located or positioned between the intermediate portion 114 and the second portion 110.

The first portion 108 of the arm 104 and the second portion 110 may be substantially parallel to one another, or alternatively, may be positioned at converging or diverging angles with respect to one another.

The arm 104 may be made of steel, aluminum, metal, plastic, composite, or any other desired material. The arm 104 may be formed having a substantially cylindrical cross-section or may be formed have any other desired cross-sectional shape, including, but not limited to, square, rectangular, triangular, or ovular.

The arm 104 may also include a male threaded portion 118 that may extend from the first portion 108 of the arm 104. The male threaded portion 118 may be designed and configured to be threaded into a female threaded portion 120 of the handle 102. Although any other desired coupling mechanism may be used to couple the arm 104 to the handle 102.

The female threaded portion 120 of the handle 102 may be located at a first end 122 of the handle 102. The first end 122 of the handle 102 may be located substantially opposite a second end 124 of the handle 102.

In alternative embodiments the arm 104 may include a female threaded portion instead of a male threaded portion that may extend from the first portion 108 of the arm 104. In this alternative embodiment, the female threaded portion may be designed and configured to receive a male threaded portion of the handle 102. Although any other desired coupling mechanism may be used to couple the arm 104 to the handle 102.

In still further alternative embodiments, the handle 102 and the arm 104 may be permanently fixed to one another, or may be formed as a single unitary instrument.

The handle 102 may be made of steel, aluminum, metal, plastic, composite, or any other desired material. The handle 102 may be formed having a substantially cylindrical cross-section or may be formed have any other desired cross-sectional shape, including, but not limited to, square, rectangular, triangular, or ovular.

The tip 106 may include a first terminating end 122 that may include an engagement member 124. The engagement member 124 may be designed and configured to connect with the second portion 110 of the arm 104. The engagement member 124 may include a threaded portion, or any other desired mechanism for removably coupling the tip 106 to the arm 104.

The tip 106 may also include a second terminating end 126 that is substantially opposite to the first terminating end 122. The tip 106 may also include a top surface 128. The top surface 128 may extend from the second terminating end 126. The tip 106 may also include a bottom surface 130. The bottom surface 130 may also extend from the second terminating end 126.

The top surface 128 and the bottom surface 130 of the tip 106 may form an angle that may range from 10-45 degrees, or 5-60 degrees, or any other desired angle. The tip 106 may also have a thickness between the top surface 128 and the bottom surface 130 of 0-2 mm at the second terminating end 126, and may increase to a thickness of 1-5 mm of thickness between the top surface 128 and the bottom surface 130 as the tip extends toward the first terminating end 122.

The tip 106 may also include an abutment or stop 132. The abutment 132 may be adjacent to the bottom surface of the tip 130. The abutment 132 may be positioned and located substantially perpendicular to the bottom surface 130. The abutment 132 may be positioned and located at any desired angle with respect to the bottom surface 130, such as an acute angle or an obtuse angle. The abutment 132 may also form an interior or concave corner 134 with the bottom surface 130. The abutment 132 may also form an interior or concave portion that may be curved and not form a distinct corner with the bottom surface 130.

The length of the bottom surface 130 from the second terminating end 126 to the abutment may be 10-20 mm or 12-18 mm or any other desired length. The top surface 128 of the tip may have a width of 5-20 mm, or 8-15 mm or any desired width.

The tip 106 may be made of steel, aluminum, metal, plastic, composite, or any other desired material. The tip 106 may be formed having a substantially cylindrical cross-section or may be formed have any other desired cross-sectional shape, including, but not limited to, square, rectangular, triangular, or ovular.

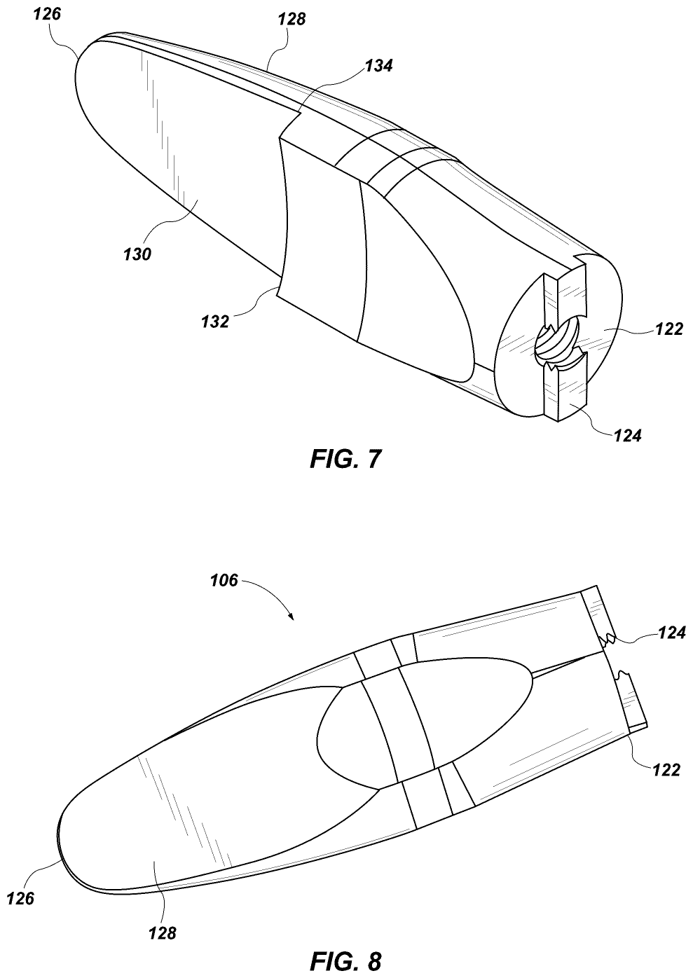

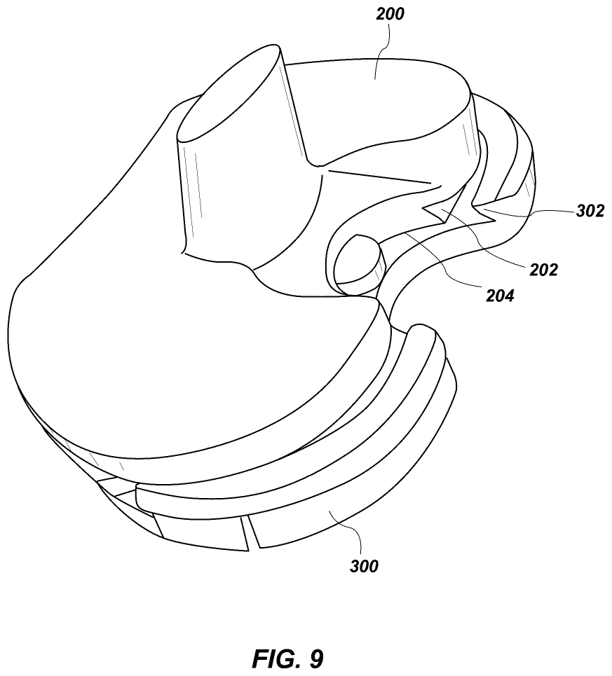

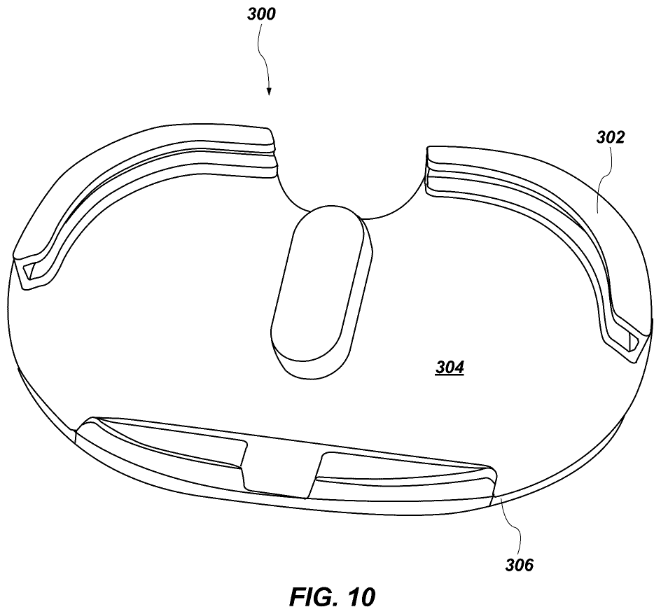

Referring to FIGS. 7-9, a user or surgeon may use the surgical instrument 100 to facilitate or aid in inserting a tibial implant 200 or other surgical implant into a cup or implant holder 300. During a surgical procedure, a surgeon may implant the implant holder 300 into a bone (not shown). Once the implant holder 300 is secured or fixed to the bone, the surgeon can insert the implant 200 into the implant holder 300.

The implant 200 may include a groove 202 that may extend around a portion of a perimeter of the implant 200. This groove 202 may be designed and configured to receive a tongue 302 formed around a portion of a perimeter of the implant holder 300.

In order to lock or secure the implant 200 into the implant holder 300, it may be beneficial if the implant 200 is inserted into the implant holder 300 at a predetermined angle with respect to the implant holder 300. As the implant 200 is inserted at the predetermined angle with the implant holder 300, the groove 202 of the implant may receive the tongue 302 of the implant holder 300. Once the tongue 302 is fully engaged or set within the groove 202 the implant 200 may be pushed down and full seated, flush, against a seating surface 304 of the implant holder 300.

In order to maintain the predetermined or desired angle between the implant 200 and the implant holder 300 during insertion of the implant 200 into the implant holder 300, the surgical tool 100 may be inserted between the implant 200 and the implant holder 300. More specifically, the second terminating end 126 of the surgical tool 100 may be inserted between the implant 200 and the implant holder 300.

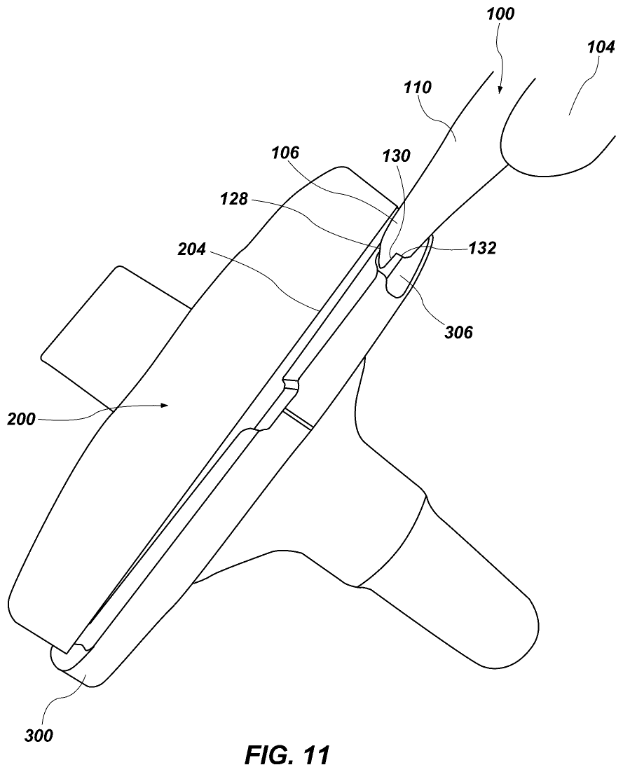

Furthermore specifically, now referring to FIGS. 6-11, the second terminating end 126 of the tip 106 of the surgical instrument 100 may be inserted between the implant 200 and the implant holder 300, such that, the bottom surface 130 of the tip 106 may be in flush contact with the seating surface 304 of the implant holder 300, and the top surface 128 of the tip 106 may be in flush contact with a bottom surface 204 of the implant 200. Additionally, the abutment 132 may be pushed into contact with an outer rim 306 of the implant holder 300, enabling a user to maintain a desired engagement depth of the tip 106 with respect to the implant holder 300. The abutment 132 also providing an additional stabilizing contact surface with the outer rim 306 of the implant holder 300.

During insertion of the implant 200, once the bottom surface 130 of the tip 106 is in flush contact with the seating surface 304 of the implant holder 300 and the abutment 132 of the tip 106 is pushed into contact with the outer rim 306 of the implant holder 300, the implant can be slid down the top surface 128 of the tip 106, the top surface 128 acting like a ramp, guiding the implant 200 into the implant holder 300.

Due to the predetermined and fixed angle between the top surface 128 and bottom surface 130 of the tip 106, the implant 200 may slide down the top surface 130 of the tip 106, at the predetermined and desired angle, so that the desired engagement between the tongue 202 and the groove 302 may be achieved. Thus the surgical tool 100 can provide a consistent, dependable angle of insertion of the implant 200 into the implant holder 300, due to the fixed, predetermined angle between the top surface 128 and bottom surface 130 of the tip 106, and the additional stabilizing contact surface provided by the contact between the abutment 132 of the tip 106 and the outer rim 306 of the implant holder 200.

Once the desired engagement has been achieved, the surgical instrument 100 may be withdrawn from contact with the implant 200 and the implant holder 300, and the implant 200 may be pushed into full, secured engagement with the implant holder 300.

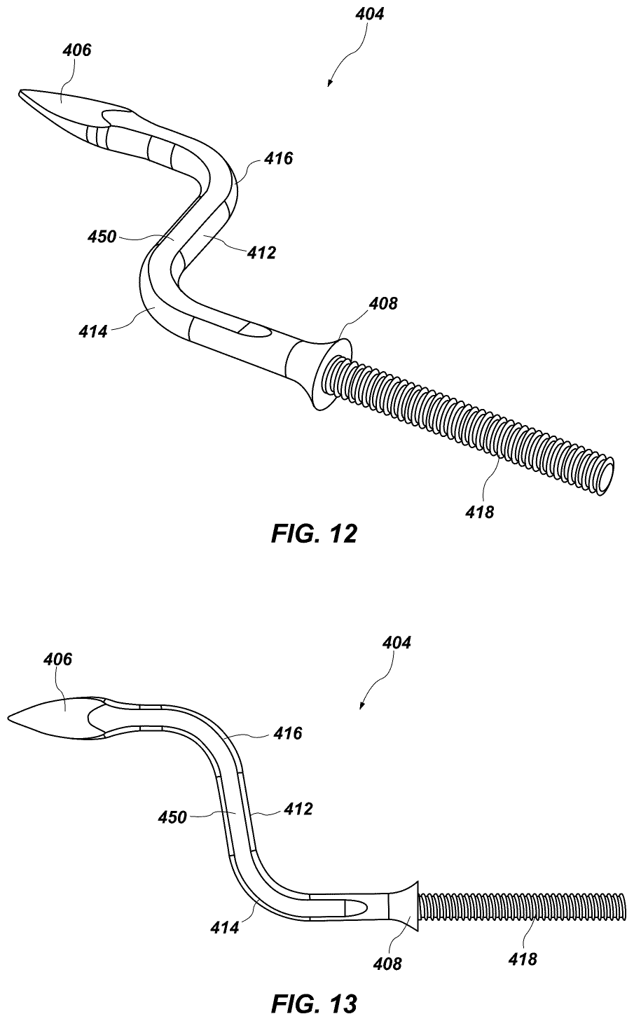

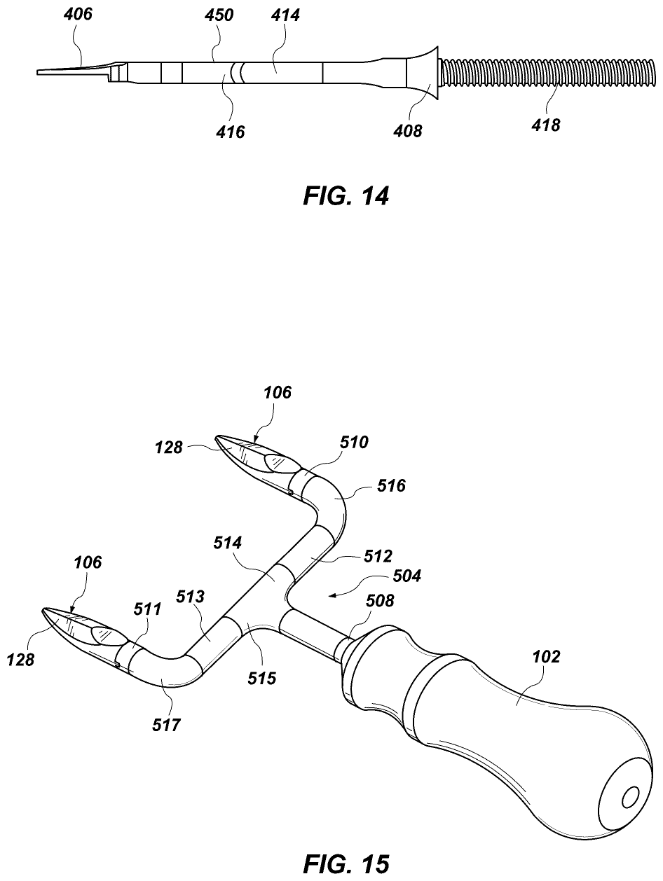

Referring to FIGS. 12-14, the surgical instrument 100 disclosed above, may include the handle 102, and another embodiment of an arm 404 and a tip 406. The arm 404 may be fixed or removably coupled to the handle 102. The tip 406 may be fixed or unitary with the arm 404 or removably coupled to the arm 404.

The arm 404 may have a first portion 408 that may removably coupled or fixed to the handle 402. The arm 404 may also have a second portion 410 that may be removably coupled or fixed to the tip 406. The arm 404 may also include an intermediate portion 412 that is positioned or located between the first portion 408 and the second portion 410.

The arm 404 may include a first bend 414 located or positioned between the first portion 408 and the intermediate portion 414. The arm 404 may also include a second bend 416 located or positioned between the intermediate portion 414 and the second portion 410.

The first portion 408 of the arm 404 and the second portion 410 may be substantially parallel to one another, or alternatively, may be positioned at converging or diverging angles with respect to one another.

The arm 404 may be made of steel, aluminum, metal, plastic, composite, or any other desired material. The arm 404 may be formed having a substantially cylindrical cross-section or may be formed have any other desired cross-sectional shape, including, but not limited to, square, rectangular, triangular, or ovular.

The arm 404 may also include a male threaded portion 418 that may extend from the first portion 408 of the arm 404. The male threaded portion 418 may be designed and configured to be threaded into a female threaded portion 120 of the handle 102. Although any other desired coupling mechanism may be used to couple the arm 404 to the handle 102.

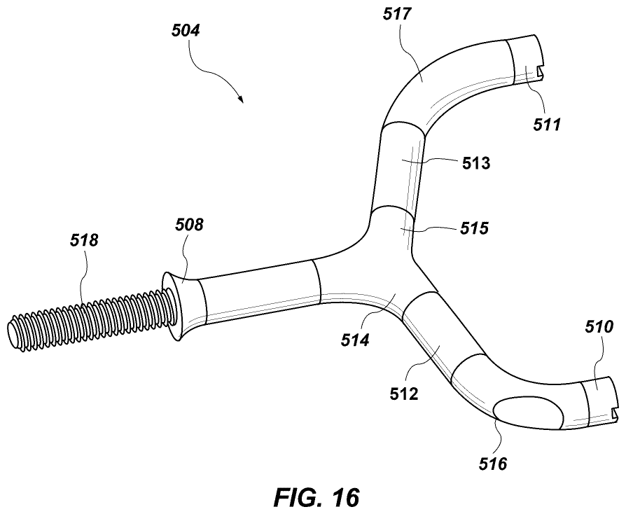

Referring to FIGS. 15 and 16, the surgical instrument 100 disclosed above, may include the handle 102, and another embodiment of an arm 504 and a pair of tips 106. The arm 504 may be fixed or removably coupled to the handle 102. The tips 506 may be fixed or unitary with the arm 504 or removably coupled to the arm 504.

The arm 504 may have a first portion 408 that may be removably coupled or fixed to the handle 102. The arm 504 may also have a second portion 510 that may be removably coupled or fixed to the tip 506. The arm 504 may also include an first intermediate portion 512 that is positioned or located between the first portion 408 and the second portion 510. The arm 504 may also have a third portion 511 that may be removably coupled or fixed to corresponding tips 106. The arm 504 may also include a second intermediate portion 513 that is positioned or located between the first portion 508 and the third portion 511.

The arm 504 may include a first bend 514 located or positioned between the first portion 508 and the first intermediate portion 514. The arm 504 may also include a second bend 516 located or positioned between the first intermediate portion 514 and the second portion 510.

The arm 504 may include a third bend 515 located or positioned between the first portion 508 and the second intermediate portion 515. The arm 504 may also include a fourth bend 517 located or positioned between the second intermediate portion 515 and the third portion 511.

The first portion 508 of the arm 504 and the second portion 510 and the third portion 511 may be substantially parallel to one another, or alternatively, may be positioned at converging or diverging angles with respect to one another.

The arm 504 may be made of steel, aluminum, metal, plastic, composite, or any other desired material. The arm 404 may be formed having a substantially cylindrical cross-section or may be formed have any other desired cross-sectional shape, including, but not limited to, square, rectangular, triangular, or ovular.

The arm 504 may also include a male threaded portion 518 that may extend from the first portion 508 of the arm 504. The male threaded portion 518 may be designed and configured to be threaded into a female threaded portion 120 of the handle 102. Although any other desired coupling mechanism may be used to couple the arm 504 to the handle 102.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed:

1. A surgical instrument, comprising:
a handle;
an elongate member extending from the handle, the handle coupled to the elongate member at a first portion of the elongate member;
a tip extending from the elongate member, the tip coupled to a second portion of the elongate member;
wherein the tip includes a top surface and a bottom surface extending from a terminating end of the tip, wherein the top surface and the bottom surface form a converging angle; and
wherein the tip also includes an abutment that is adjacent to the bottom surface.

2. The surgical instrument of claim 1, wherein the abutment is substantially perpendicular to the bottom surface.

3. The surgical instrument of claim 1, wherein the elongate member includes a first portion, a second portion, a first bend and a second bend, wherein the first bend and the second bend are located between the first portion and the second portion.

4. The surgical instrument of claim 1, wherein the elongate member includes first and second bends.

5. The surgical instrument of claim 1, wherein the abutment and the bottom surface of the tip form an interior corner.

6. The surgical instrument of claim 1, wherein the converging angle formed by the top surface and the bottom surface is between 10-45 degrees.

7. The surgical instrument of claim 1, wherein the bottom surface of the tip has a length of 10-20 mm.

8. The surgical instrument of claim 1, wherein the handle and the elongate member are formed as a single unitary piece.

9. The surgical instrument of claim 1, wherein the elongate member includes a plurality of tips.

10. The surgical instrument of claim 9, wherein the elongate member includes four bends.

11. The surgical instrument of claim 1, wherein the elongate member is an arm.

12. A surgical instrument, comprising:
a handle;
a tip connected to the handle;
wherein the tip includes a top surface and a bottom surface extending from a terminating end of the tip, wherein the top surface and the bottom surface form a converging angle; and
wherein the tip also includes an abutment that is substantially perpendicular to the bottom surface.

13. The surgical instrument of claim 12, further comprises:
an elongate member extending from the handle, the handle coupled to the elongate member at a first portion of the elongate member.

14. The surgical instrument of claim 13, wherein the elongate member includes a first portion, a second portion, a first bend and a second bend, wherein the first bend and the second bend are located between the first portion and the second portion.

15. The surgical instrument of claim 13, wherein the elongate member includes first and second bends.

16. The surgical instrument of claim 12, wherein the abutment and the bottom surface of the tip form an interior corner.

17. The surgical instrument of claim 12, wherein the converging angle formed by the top surface and the bottom surface is between 10-45 degrees.

18. The surgical instrument of claim 12, wherein the bottom surface of the tip has a length of 10-20 mm.

19. The surgical instrument of claim 13, wherein the handle and the elongate member are formed as a single unitary piece.

20. The surgical instrument of claim 13, wherein the elongate member includes a plurality of tips.

21. The surgical instrument of claim 20, wherein the elongate member includes four bends.

22. The surgical instrument of claim 13, wherein the elongate member is an arm.

23. A surgical instrument, comprising:
a handle;
an elongate member extending from the handle, the handle coupled to the elongate member at a first portion of the elongate member, wherein the elongate member includes a first portion, a second portion, a first bend and a second bend, wherein the first bend and the second bend are located between the first portion and the second portion;
a tip extending from the elongate member, the tip coupled to a second portion of the elongate member;
wherein the tip includes a top surface and a bottom surface extending from a terminating end of the tip; and
wherein the tip also includes an abutment that is adjacent to the bottom surface.

24. The surgical instrument of claim 23, wherein the top surface and the bottom surface of the tip form a converging angle.

25. The surgical instrument of claim 23, wherein the abutment is substantially perpendicular to the bottom surface.

26. The surgical instrument of claim 24, wherein the elongate member includes first and second bends.

27. The surgical instrument of claim 23, wherein the abutment and the bottom surface of the tip form an interior corner.

28. The surgical instrument of claim 23, wherein the converging angle formed by the top surface and the bottom surface is between 10-45 degrees.

29. The surgical instrument of claim 23, wherein the bottom surface of the tip has a length of 10-20 mm.

30. The surgical instrument of claim 24, wherein the handle and the elongate member are formed as a single unitary piece.

31. The surgical instrument of claim 24, wherein the elongate member includes a plurality of tips.

32. The surgical instrument of claim 31, wherein the elongate member includes four bends.

33. The surgical instrument of claim 23, wherein the elongate member is an arm.

34. A surgical instrument, comprising:
a handle;
an elongate member extending from the handle, the handle coupled to the elongate member at a first portion of the elongate member;
a tip extending from the elongate member, the tip coupled to a second portion of the elongate member;
wherein the tip includes a top surface and a bottom surface extending from a terminating end of the tip, wherein the top surface and the bottom surface form a converging angle; and
wherein the tip also includes an abutment that is adjacent to the bottom surface, wherein the abutment is substantially perpendicular to the bottom surface;
wherein the elongate member includes a first portion, a second portion, a first bend and a second bend, wherein the first bend and the second bend are located between the first portion and the second portion;
wherein the abutment and the bottom surface of the tip form an interior corner;
wherein the converging angle formed by the top surface and the bottom surface is between 10-45 degrees;
wherein the bottom surface of the tip has a length of 10-20 mm; and
wherein the handle and the elongate member are formed as a single unitary piece.
